(19) United States
(12) Patent Application Publication
(10) Pub. No.: US 2005/0064408 A1
(43) Pub. Date: Mar. 24, 2005

(54) Method for gene mapping from chromosome and phenotype data

(76) Inventors: Petteri Sevon, Helsinki (FI); Hannu T., T. Toivonen, Helsinki (FI); Vesa Ollikainen, Helsinki (FI)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/480,325

(22) PCT Filed: Jun. 11, 2002
(86) PCT No.: PCT/FI02/00504

(30) Foreign Application Priority Data
Jun. 13, 2001 (FI) 20011250

(51) Int. Cl. C12Q 1/68; G06F 19/00; G01N 33/48; G01N 33/50

(52) U.S. Cl. 435/6; 702/20

(57) ABSTRACT

The present invention relates to a method for gene mapping from chromosome and phenotype data, which utilizes linkage disequilibrium between genetic markers mᵢ, which are polymorphic nucleic acid or protein sequences or strings of single-nucleotide polymorphisms deriving from a chromosomal region. The method according to the invention is based on discovering and assessing tree-like patterns in genetic marker data. It extracts, essentially in the form of substrings and prefix trees, information about the historical recombinations in the population. This infor-mation is used to locate fragments potentially inherited from a common diseased founder, and to map the disease gene into the most likely such fragment. The method measures for each chromosomal location the disequilibrium of the prefix tree of marker strings starting from the location, to assess the distribution of disease-associated chromosomes.

### FIELD OF THE INVENTION

The present invention relates to a method for gene mapping from chromosome and phenotype data, which utilizes linkage disequilibrium between genetic markers mᵢ, which are polymorphic nucleic acid or protein sequences or strings of single-nucleotide polymorphisms deriving from a chromosomal region.

### BACKGROUND OF THE INVENTION

Gene mapping aims at discovering a statistical connection from a particular disease or trait to a narrow region in the genome probably containing a gene that affects the trait. In particular, the discovery of new disease susceptibility genes can have an immense importance for human health care. The gene and the proteins it produces can be analyzed to understand the disease causing mechanisms and to design new medicines. Further, gene tests on patients can be used to assess individual risks and for preventive and individually tailored medications. Obviously, gene mapping is receiving increasing interest among medical industry.

Genetic markers along chromosomes provide data that can be used to discover associations between patient phenotypes (e.g., diseased vs. healthy) and chromosomal regions (i.e., potential disease gene loci). The growing number of available genetic markers, anticipated to reach hundreds of thousands in the next few years, offers new opportunities but also amplifies the computational complexity of the task.

Human genome sequencing efforts, the first ones now almost complete, read the full human DNA sequence. There are methods for recognizing where there are genes in the sequence—the number of which is currently estimated to be around 30,000. However, we lack methods for deriving the function of a gene from the sequence information. Gene mapping approaches this problem for one disease at a time. It aims at discovering areas in the genome—hopefully small—that have a statistical connection to a given trait, thus narrowing down the area to be analyzed with expensive laboratory methods.

A typical setting for gene mapping is a case-control study of some chromosome of diseased and healthy individuals. Instead of looking at the DNA of the whole chromosome, only certain marker segments distributed along the chromosome are considered. By the analysis of similarities within the disease-associated chromosomes on one hand and the differences between the disease-associated and control chromosomes on the other, one can try to locate likely areas for a gene that predisposes people to the disease analyzed.

The overall goal of the method according to the invention is to locate a disease-susceptibility gene for a given disease. In gene mapping, the aim is to identify a narrow chromosomal region within which the gene is likely to be; this area can then be analyzed in more detail with laboratory tools. We next briefly review the genetic background; without loss of generality, we restrict the discussion in this paper to one chromosome.

Marker Data

A genetic marker is a short polymorphic region in the DNA, denoted here by M1, M2, . . . . The different variants of DNA that different people have at the marker are called alleles, denoted in our examples by 1, 2, 3, . . . . The number of alleles per marker is small: typically less than ten for microsatellite markers, and exactly two for single nucleotide polymorphisms (SNP). The collection of markers used in a particular study is its marker map, and the corresponding alleles in a given chromosome constitute its haplotype (FIG. 1). It is a major task of a gene mapping study to design the marker map and to obtain the haplotype data. That is where we start, and for the purposes of this paper the input data consists of haplotypes of diseased and control persons—or, in computer science terms, aligned allele strings, classified to positive and negative examples.

Linkage Disequilibrium

All the current carriers of a disease-susceptibility gene have inherited it from a founder who introduced the gene mutation to the population. If there has been only one or few such founders, then many of the current carriers are related, may share some segments of the chromosome, and lend themselves to gene mapping studies. In particular, segments from the mutation carrying founder chromosomes are over-represented among the affected at mutation locus. Relatively young (e.g. 1000 years) population isolates are promising sources of data in this respect: disease-susceptibility genes may have been introduced by one or two founders only, and the gene may be over-represented in the population. Kainuu region in eastern Finland is an example of such a fruitful area for genetic studies.

If there are conserved regions at the mutation locus, then it can be possible to observe linkage disequilibrium (LD), or non-random association between nearby markers (FIG. 2). There are severe statistical problems, however, in observing LD. Mutation carriers often only have a higher risk of being diseased than non-carriers, and in a case-control study both groups can be mixes of carriers and non-carriers. Further on, since the selection of patients is more or less random, and the whole coalescence process leading to LD is stochastic, it is a challenge to recognize LD and the DS gene location from all the noise.

Gene Mapping

In diseases with a reasonable genetic contribution, and especially in population isolates, affected individuals are likely to have higher frequencies of certain alleles and haplotype patterns near the DS gene than control individuals. This is the starting point of LD-based mapping methods: where does the set of affected chromosomes show linkage disequilibrium? The problem is far from trivial, however. The coalescence process is stochastic; mutation carriers often only have a higher risk of being diseased than non-carriers, and in a case-control study both groups are usually mixes of carriers and non-carriers; and finally, there is missing information and haplotyping ambiguities.

Most current gene mapping methods based on linkage disequilibrium look just at individual markers or neighboring markers, measure their association to the disease status, and predict the gene locus to be co-located with the strongest association. However, since different mutation carriers share different segments, there is no single marker or pattern that is representative of the shared segments.

In the recent years, several statistical methods have been proposed to detect LD (Terwilliger 1995, Devlin et al. 1996, Lazzeroni 1998, Service et al. 1999, McPeek et al. 1999). The emphasis has been on fairly involved statistical models of LD around a DS gene. They model whole recombination histories and some are robust to high levels of heterogeneity. On the other hand, the models are based on a number of assumptions about the inheritance model of the disease and the structure of the population, which may be misleading for the statistical inference. The methods tend to be computationally heavy and therefore better suited for fine mapping than genome screening.

Haplotype Pattern Mining or HPM (Toivonen et al. 2000a, Toivonen et al. 2000b) is based on analyzing the LD of sets of haplotype patterns, essentially strings with wildcard characters. The method first finds all haplotype patterns that are strongly associated with the disease status, using ideas similar to the discovery of association rules (Agrawal et al. 1993, Agrawal et al. 1996). Since the patterns may contain gaps they can account for some missing and erroneous data. In the second step, each marker is ranked by the number of patterns that contain it. Either this score is used as a basis for the prediction or, preferably, a permutation test is used to obtain marker-wise p values. HPM has been extended for detecting multiple genes simultaneously (Toivonen et al. 2000b) and to handle quantitative phenotypes and covariates (Sevon et al. 2001).

Nakaya et al (Nakaya et al. 2000) investigate the effect of multiple separate markers, each one thought to correspond to one gene, on quantitative phenotypes. Their work is a generalization of the LOD score to multiple loci, and it does not handle haplotype patterns.

An alternative approach for LD-based mapping is linkage analysis. The idea is to analyze family trees, and to find out which markers tend to be inherited to offspring in conjunction with the disease. Linkage analysis does not rely on common founders, so in that respect it is more widely applicable than LD-based methods. The downside is that estimates are rough (due to the smaller effective number of meiosis sampled), and that collecting information from larger families is more difficult and expensive.

Transmission/disequilibrium tests (TDT) (Spielman et al. 1993) are an established way of testing association and linkage in a sample where linkage disequilibrium exists between the mutation locus and nearby marker loci. TDT detects deviations between observed and expected counts for each allele transmitted from heterozygous parents to affected offspring.

Single permutation tests have been used in mapping studies before (Churchill and Doerge 1994, Laitinen et al. 1997, Long and Langley 1999). However, if more complex data is to be analyzed, these single permutation tests are too expensive and computationally very ineffective and even inoperative.

Genetic markers provide an economical, sparse view of chromosomes. Even sparsely located markers can be very informative: given an ancestor with a disease gene, the descendants that inherit the gene are also likely to inherit a string of alleles of nearby markers. The exact probability of inheriting any combination of markers depends on the gene location with respect to the markers, the population history or the coalescence history, and marker mutations; all of these are unknown. There is a continuous need for more effective gene mapping methods.

The object of the present invention is to provide a novel method for gene mapping from chromosome and phenotype data. The method according to the invention considers the recombination histories—sort of family trees—that are likely to have caused the observed trees of patterns. The disease susceptibility (DS) gene is then predicted to be where the strongest genetic contribution is visible in the trees. The contributions of the method according to the invention are:
(1) a novel approach to gene mapping using tree patterns,
(2) an efficient algorithm for generating and testing tree patterns,
(3) a method for the estimation of statistical significance of individual findings as well as the whole process, based on multiple permutations but carried out at the cost of a single permutation.

### SUMMARY OF THE INVENTION

It is an object of the present invention to provide a method for gene mapping from chromosome and phenotype data, which utilizes linkage disequilibrium between genetic markers mᵢ, which are polymorphic nucleic acid or protein sequences or strings of single-nucleotide polymorphisms deriving from a chromosomal region. The method of the invention comprises steps of
i) identifying a prefix tree T based on the observed haplotypes at a number of locations of a chromosome,
ii) evaluating each prefix tree T by its genetic and statistical feasibility, assuming that the gene was close to the root of the tree, and thus determining a score for each prefix tree T,
iii) predicting the area for the location of the gene as a function of the score determined in the step (ii).

The present invention is now explained in detail by referring to the attached figures and examples. These examples are only used to show some of the embodiments and are not intended to limit the scope of the invention.

### BRIEF DESCRIPTION OF THE DRAWINGS

FIG. 1. A marker map of ten markers and a sample haplotype consisting of alleles in adjacent markers.

FIG. 2. A carrier of the ancestral mutation has inherited founder alleles around the disease locus. These alleles are similar to those of the ancestral chromosome in generation 0. Due to the common inherited segment, many of the contemporary mutation carriers are expected to share alleles in the markers around the mutation, but the length of the shared haplotype varies.

FIG. 3. A possible coalescence tree at the fourth marker for the three observed haplotypes at the bottom level. Internal nodes correspond to recurrent substrings. An alternative coalescence tree would have ---344- instead of -1234-- at the second level.

FIG. 4. An illustration of the tree structure in a string-sorted set of haplotypes to the right from the location pointed by the arrow.

FIG. 5. Analysis of the performance of TreeDT. A: Gene localization power with different values of A, the proportion of disease-associated chromosomes that actually carry the mutation. B: Gene localization power with different numbers of subtrees (method parameter) and different numbers of founders (population parameter). C: Classification accuracy for the existence of a disease susceptibility gene.

FIG. 6. Comparison of the gene localization performance of TreeDT, HPM, multipoint TDT (m-TDT), and TDT. A: The baseline test setting. B: The baseline setting with three founders. C: The baseline setting with 5% missing data.

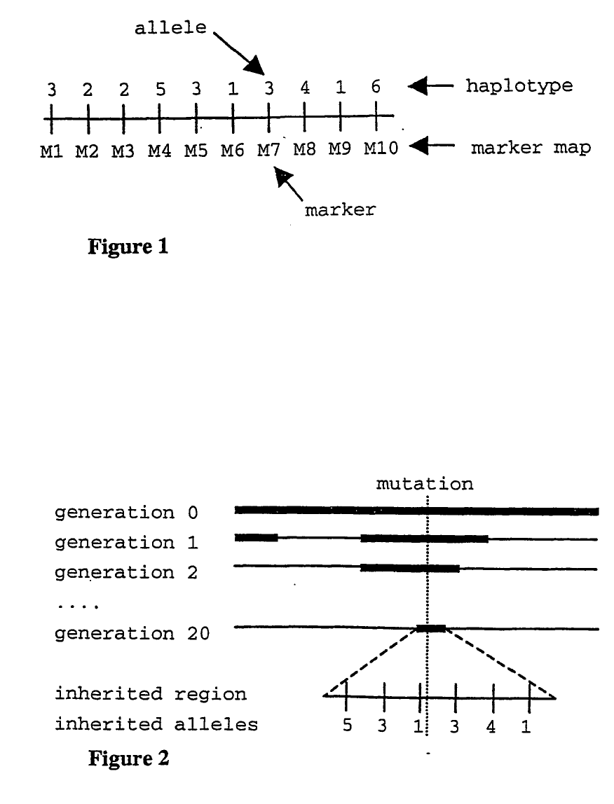

### DETAILED DESCRIPTION OF THE INVENTION

It is an object of the present invention to provide a method for gene mapping aiming to discover a gene region affecting a certain trait using chromosome data.

Empirical evaluation on a realistic, simulated data shows that the method according to the invention is competitive with other recent data mining based methods, and clearly outperforms more traditional methods. Our experiments, explained later, show that the method according to the invention, TreeDT, is effective in extreme conditions typical for current mapping problems: with lots of noise (only 10-20% of affected chromosomes carry the mutation, lots of missing data) and with small sample sizes (200 affected and 200 control chromosomes). However, the highest potential of the method according to the invention lies in the data intensive tasks of future—such as genome scanning with larger samples and larger number of markers—due to its low computational complexity.

In comparison to state of the art methods, TreeDT is most competitive. In terms of gene localization accuracy, it gave best results in the case of multiple founders and demonstrated good robustness with respect to missing data. Unlike the compared methods, TreeDT can be used to predict whether a gene is present at all or not. Finally, in comparison to its closest competitor, HPM, TreeDT has much smaller computational cost. An additional advantage of TreeDT is that it has only one input parameter, the (maximum) number of deviant subtrees, whereas for HPM one has to set several more or less arbitrary thresholds.

Method

For any pair of chromosomes in the sample there has been a common origin in the population history, an ancestral chromosome at which their paths have diverged. Due to recombinations different parts of chromosomes have different histories. At any given location the chromosomes in the sample and their most recent common origins form a coalescence tree. In the coalescence tree for the DS gene location, all the chromosomes in one or more subtrees carry the DS mutation, and we should observe excess of disease-associated haplotypes as the leaves of these subtrees. The closer the tree is located to the DS gene, the more and larger subtrees are identical to those in the tree at the DS gene location.

Based on the observed haplotypes, the method of the invention defines a prefix tree estimating the most likely coalescence tree at a number of locations along the analyzed chromosome, and then assesses the subtree clustering of disease-associated haplotypes in these trees.

It is a further object of this invention to provide a novel tree disequilibrium test, intended for predicting DS gene locations in the method of the invention. The vicinity of the location for which the test gives the lowest p value is the most likely candidate area for the DS gene location. The method also calculates the corrected overall p value for the best finding. This p value can be used for predicting whether the chromosome carries a DS gene.

Further, a method for the estimation of statistical significance of individual findings as well as the whole process, based on multiple permutations but carried out at the cost of a single permutation, is provided.

Haplotype Prefix Trees

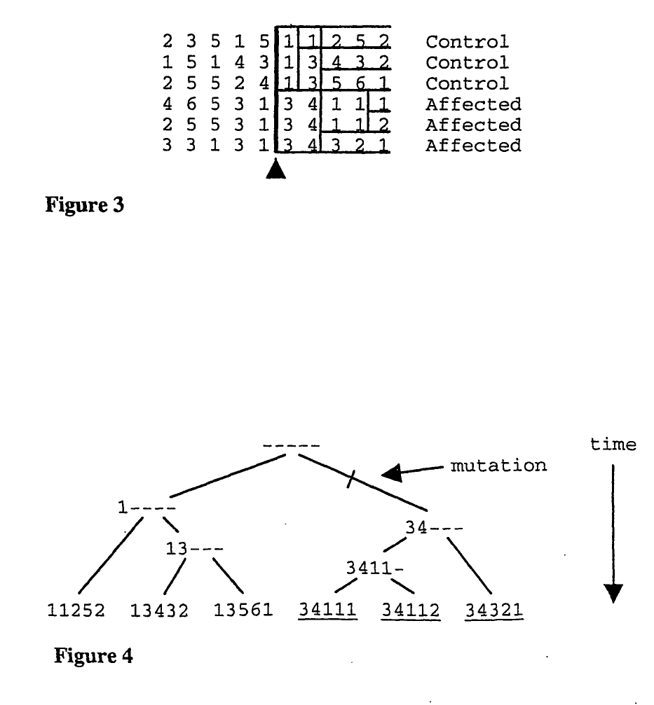

The subsumption relation of the substrings overlapping a given location forms a directed acyclic graph (DAG). The tree structures obtainable by pruning the DAG may be considered as possible coalescence trees at the location, as shown in FIG. 3, with the following exceptions: 1) The order of nodes may differ from that in the true coalescence tree, e.g. -34-- might actually be a more recent node than --1234--. However, because the expected length of the shared region of two chromosomes decreases monotonically as the time from their divergence increases, it is easy to see that the order given by subsumption is the most likely one. 2) Because haplotypes may also share a substring by chance, the internal nodes may represent a combination of nodes in true coalescence tree. The upper nodes of the coalescence tree must be very old and the corresponding shared chromosomal regions extremely short, and therefore it is very likely that a large number of coalescence nodes is contained in the empty substring root. On the other hand the younger coalescence nodes with shared regions spanning over several markers are more likely to have one-to-one correspondence with observed recurrent substrings.

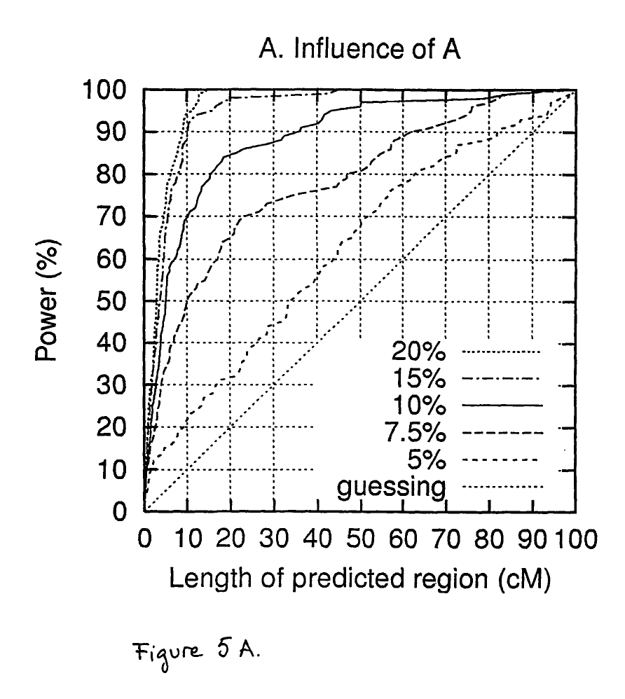

Instead of considering alternative coalescence trees leading to the same observed haplotypes, the method of the invention uses the unique haplotype prefix tree as a canonical representation of such set of coalescence trees. An example of a prefix tree is shown in FIG. 4. The method of the invention builds the prefix trees between each pair of consecutive markers and tests their disequilibrium.

Tree Disequilibrium Test

According to one embodiment of the invention, the prefix tree T is tested by the tree disequilibrium test (TreeDT) testing the alternative hypothesis The distribution of the disease-association statuses deviates in some subtrees of T from the overall distribution of statuses against the null hypothesis The disease-association statuses are randomly distributed in the leaves of T. TreeDT identifies the subtree set in which the observed status distribution deviates most from the expectation under the null hypothesis, and returns the significance of the deviation as a p value. TreeDT takes the maximum number of deviant subtrees as a parameter. In principle there is no need to set an upper limit for the subtree count, but whenever LD-mapping is applicable, the majority of the mutation carriers is concentrated in a only few such subtrees in which the shared region is long enough to identify a deviant substring. In the experiments for this paper we use an upper limit of 6 subtrees.

For measuring the disequilibrium of a tree, we use a variant of the Z test. The test statistic Zₖ for a tree with k deviant subtrees T₁, . . . , Tₖ is

$${Z}_{k} = {\sum }_{i = 1}^{k} \frac{{a}_{i} - {n}_{i} p}{\sqrt{{n}_{i} p ( 1 - p )}} ,$$

where aᵢ is the number of disease-associated haplotypes and nᵢ the total number of haplotypes in subtree TᵢεS, and p is the proportion of disease-associated haplotypes in the sample. The score measures the distance of the observed number of disease-associated chromosomes (aᵢ) from the expectation (nᵢp) in standard deviations (the square root of nᵢp(1−p)), under the assumption of binomial distribution with parameters nᵢ and p. We use a one-tailed test, since we are interested only in subtrees in which the proportion of disease-associated haplotypes is greater than expected.

We could use a 2×(k+1) χ²-statistic as a measure of deviation for a given subtree set S. The χ²-statistic, however, is not easily maximized in the space of all possible subtree sets and is therefore not a very practical choice.

Zₖ can be efficiently maximized simultaneously for all k using a recursive algorithm, as shown in the Algorithms section.

TreeDT takes the maximum number of deviant subtrees as a parameter. In principle there is no need to set an upper limit for the subtree count, but whenever LD-mapping is applicable, the majority of the mutation carriers is concentrated in only few such subtrees in which the shared region is long enough to identify a deviant substring. In the experiments for this paper we use an upper limit of 6 subtrees.

Significance Tests with Nested Permutations

Zₖ is a measure for the disequilibrium of a given tree, corresponding to a certain location in the chromosome, with given k deviant subtrees. Given a tree, TreeDT finds for each k the set S of subtrees that maximizes Zₖ. In order to find the best k for the given tree, simple maximization is not possible. Since the statistics for different degrees of freedom k are not comparable, TreeDT estimates the p value for each maximized Zₖ (under the null hypothesis of random distribution of disease status). Because the distribution of the maximized Zₖ is very complex and dependent on the tree structure, p values are estimated by a permutation test.

In order to get a single p value for the disequilibrium at a given location, we need to combine the information from the trees to the left and to the right of the location. As a combined measure we use the product of the lowest p value over all k from each side. Again, since the measures are not necessarily directly comparable, a new p value for the combination is estimated. The results are now comparable between different locations.

The output of TreeDT is essentially the p value ranked list of locations. A point prediction for the gene location is obtained by taking the best location; a (potentially fragmented) region of length l is obtained by taking best locations until a length of l is covered.

Since multiple locations are tested for a p value, and also since the p values at nearby locations are not independent, a direct link between the p value and the probability that the gene is indeed close to the location can not be established. The p values are used simply as a method of ranking the locations.

However, a single corrected p value for the best finding can be obtained with a third test using the lowest local p value as the test statistic. This p value can also be used to answer the question whether there is a gene in the investigated are in the first place or not.

All these three nested p value tests (for each tree and k, for each location, for the best location) can be carried out efficiently at the cost of a single test. Table 1 summarizes the three levels of the nested test.

TABLE 1
A summary of the permutation test procedure.

| | For | | |
|---|---|---|---|
| Level | Each | Test statistic | Result |
| 1 | (T, k) | max Zₖ(S, T) over all | p(T, k) |
| | | S ∈ SubtreeSets(T) | |
| 2 | t | min p(T₁, k₁) p(T₂, k₂) | p(t), the p value of the tree disequi- |
| | | over all k₁, k₂ | librium test for the pair |
| | | | t = (T₁, T₂) of left- and right-side |
| | | | trees rooted at the same location |
| 3 | | min p(t) over all t | p, the corrected overall p value |

Algorithms
Constructing the Haplotype Prefix-Trees

The haplotype prefix-trees to the left and right from each analyzed location can be efficiently identified using a string-sorting algorithm. The algorithm produces as intermediate results for each marker the sorted list of the partial haplotypes to the right from the marker. All the right-side trees can be easily derived from these intermediate lists, because the haplotypes belonging to a single node form a continuous block in the sorted list. The left-side trees can be identified similarly by sorting the inverted haplotypes. The computational cost of constructing the trees is negligible compared to the cost of the permutation test procedure.

The same process can also be used to enumerate all the recurrent substrings, or all the closed substrings. A substring s is closed, if and only if none of its superstrings match all the same haplotypes than s. The nodes in the right-side prefix trees have one-to-one correspondence to recurrent substrings starting at the same marker. Nodes that are to be split in the next step of the sort algorithm correspond to closed substrings.

An Algorithm for Maximizing the Tree Disequilibrium Statistic

It is essential that the time-complexity of the algorithm for maximizing the Z-values is as low as possible, because it must be executed for each tree location and permutation in turn. The key observation is that if S is the set of k deviant subtrees of T with the greatest value of Zₖ, T′ is a subtree of T, and S′⊂S is a set of m subtrees in T′, then S′ has the maximum value of Zₘ in T′. Also, if S=S₁∪ . . . ∪Sₙ, and k is the subtree count in S, and kᵢ is the subtree count in Sᵢ, then

$${Z}_{k} ( S ) = {\sum }_{i}^{} {Z}_{{k}_{i}} ( {S}_{i} ) .$$

These observations lead us to the following recursive algorithm that propagates the locally maximized Z-values upwards in the tree:
Input: A haplotype prefix tree T
Output: Maximum values of Zₖ in the tree T for each k
Call Maximize(T)
Maximize(T):
If T is not a leaf:
1. For each immediate subtree Tᵢ of T: Recursively call Maximize(Tᵢ).
2. For each k: calculate the maximum value Z_{MAX, k}(T) for Zₖ(S,T) over all S that can be obtained by combining subtree sets from each subtree Tᵢ of T.
3. Calculate Z₁ for T. If Z₁>Z_{MAX, 1}(T) then set Z_{MAX, 1}(T): =Z₁.
If T is a leaf, then set Z_{MAX, 1}(T): =0.
Step 2 can be further refined:
2.1 Set Yₖ: =0 and Z_{MAX, k}(T): =0 for all k, 1≦k≦n, where n is the number of leaves in T.
2.2 For each subtree T′ of T:
2.2.1 For each pair (i,j), 1≦i≦p and 1≦j≦q, where p is the number of leaves in T′ and q is the total number of leaves in all the subtrees processed prior to T′:
   If Z_{MAX, i}(T′)+Yⱼ>Z_{MAX, i+j}(T), then set Z_{MAX, i+j}(T): =Z_{MAX, i}(T′)+Yⱼ.
2.2.2 For each k, 1≦k≦p:
   If Z_{MAX, k}(T′)>Z_{MAX, k}(T), then set Z_{MAX, k}(T): =Z_{MAX, k}(T′).
2.2.3 For each k, 1≦k≦p+q:
   If Z_{MAX, k}(T)>Yₖ(T), then set Yₖ(T)=Z_{MAX, k}(T)

The time complexity of the algorithm is O(n²) (proof omitted), where n is the number of leaves in the tree i.e. the number of haplotypes in the data set. By setting an upper limit k for the size of the subtree sets, the average time complexity can be reduced to O(n) with a constant coefficient proportional to k², k being typically small, ≦10.

An Efficient Algorithm for Multiple Nested Permutation Tests

The straightforward algorithm for a three-level nested permutation test using nested loops would have time complexity of O(n³qr), where n is the number of permutations at each level, q is the time complexity of maximizing the Zₖ-statistic for all k, and r is the number of tested locations in the chromosome. The test would be intractable already with rather small permutation counts. However, the time complexity can be drastically reduced using the same set of permutations at each level of the test and thus only maximizing the Zₖ-values n instead of n³ times for each location.
1. Compute Z_{MAX, k}(T)=max Zₖ(T,S) for each subtree count k and each coalescence tree T over all SεSubtreeSets(T).
2. Randomly generate n+1 permutations of disease-association statuses for the haplotypes and for each permutation i and (T,k): compute Z_{MAX, k}(i,T)=max Zₖ(i,T,S) over all SεSubtreeSets(T).
   //Level 1
3. For each (T,k):
3.1 Calculate a p value p(T,k) by comparing Z_{MAX, k}(T) to Z_{MAX, k}(i,T), 1≦i≦n.
3.2 For each permutation i: calculate a p value p(i,T,k) by comparing Z_{MAX, k}(i,T) to all Z_{MAX, k}(j,T), j≠i.
   //Level 2
4. For each pair of opposed trees rooted at the same location t=(T₁,T₂):
4.1 Choose p_{MIN}(t)=min p(T₁,k₁)p(T₂,k₂) over all k₁, k₂
4.2 For each permutation i: choose p_{MIN}(i,t)=min p(i,T₁,k₁)p(i,T₂,k₂) over all k₁, k₂.
4.3 Calculate a p value p(t) by comparing p_{MIN}(t) to p_{MIN}(i,t), 1≦i≦n.
4.4 For each permutation i: calculate a p value p(i,t) by comparing p_{MIN}(i,t) to all p_{MIN}(i,t), j≠i.
   //Level 3
5. Choose p_{MIN}=min p(t) over all t.
6. For each permutation i: choose p_{MIN}(i)=min p(i,t) over all t.
7. Calculate the overall corrected p value by comparing p_{MIN} to p_{MIN}(i), 1≦i≦n.

The time complexity of steps 3.2 and 4.4 is O(n log n) using an algorithm which first sorts the values of the test statistic for all the permutations. Step 2 predominates the time complexity of the algorithm, O(nqr), where s is the upper limit for the number of subtrees allowed in a set, q is the time complexity of maximizing the Zₖ-statistic for all k, and r is the number of tested locations in the chromosome.

Due to the finite number of permutations, the precision of the p values given by a permutation tests may not be sufficient for accurate localization. In some situations even a very large number of permutations does not produce any values for the test statistic more extreme than the observed values for several consecutive tree locations. For this purpose the p values returned by the first and second level permutation tests are determined slightly unconventionally: At level 1 we use a slightly modified version of algorithm 2 to obtain an upper bound of Zₖ for all k. At level 2 the smallest possible value for the test statistic is zero. These values correspond to p values of 1/2(n+1). The returned p value is interpolated between the p values corresponding to the next lower and higher values for the test statistic obtained by permutations. The top-level test returning the overall p value is implemented in the usual conservative manner.

### EXAMPLES

Certain embodiments and results of the present invention are described in the following non-limiting examples.

We compare TreeDT empirically to TDT, an established mapping method, and to HPM, our recent proposal based on pattern discovery. We evaluate the methods on a difficult data collection carefully simulated to resemble a realistic population isolate.

### Example 1

### Simulation of Data

We designed several different test settings, with variation in the fraction (A) of mutation carriers in the disease-associated chromosomes, in the number of founders who introduced the mutation to the population, and in the amount of missing information. For statistical analyses, we created 100 independent artificial data sets in each test setting. Great care was taken to generate realistic data by a simulation procedure that included four steps: pedigree generation, simulation of inheritance, diagnosing, and sampling.

The population pedigree was set to grow from 100 to 100,000 individuals in a period of 20 generations. In each generation, the selection of parents for each child was random, but once a couple was formed, all subsequent children allocated to either of the parents were set to be common children of the couple.

The inheritance of chromosomes within the population pedigree was simulated by first allocating a continuous chromosomal segment of 100 centiMorgans to each founder individual in generation 1.

Morgan is a unit of genetic length. 1 cM is the distance at which recombination occurs 1 out of every 100 times, about 10⁶ base pairs. Human chromosomes are roughly of 50-300 cM.

Next, the entire pedigree was traversed top-down, and, in each inheritance event, gametes were created by simulating meiosis under the assumption that the number of chiasmata in the pair of homologous chromosomes was taken from Poisson distribution with parameter one (corresponding to the genetic length of 100 cM), and their locations selected randomly. A related approach was originally presented in (Terwilliger et al., 1993).

For a baseline test setting we selected a challenging disease model where only a small proportion (A=10%) of the disease-associated chromosomes carries the disease-predisposing mutation, a complication that often is encountered in the analysis of common diseases. In the baseline setting there is one founder, and on average 3.7% of alleles are missing, making the mapping task more difficult but also more realistic.

The location of the mutation was selected randomly and independently for each of the 100 data sets produced in every setting. Each data set was in turn collected from 100 affected individuals. The length of the region to be analyzed was 100 cM. Allelic data were created using a map of 101 equidistantly spaced markers, each having 5 alleles. Both chromosomes of each affected individual in each sample were labeled disease-associated whereas the control chromosomes were constructed from the non-transmitted alleles in the parental chromosomes. Each data set thus consisted of 200 disease-associated and 200 control chromosomes

### Example 2

### Analysis of TreeDT

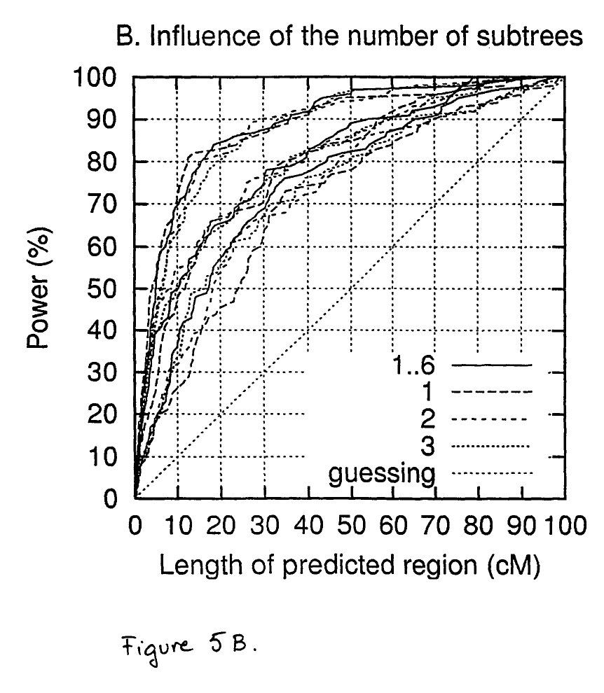

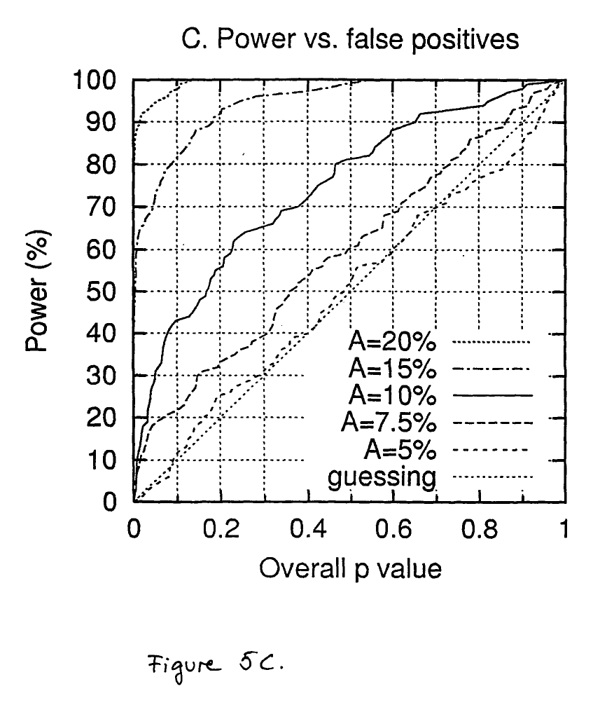

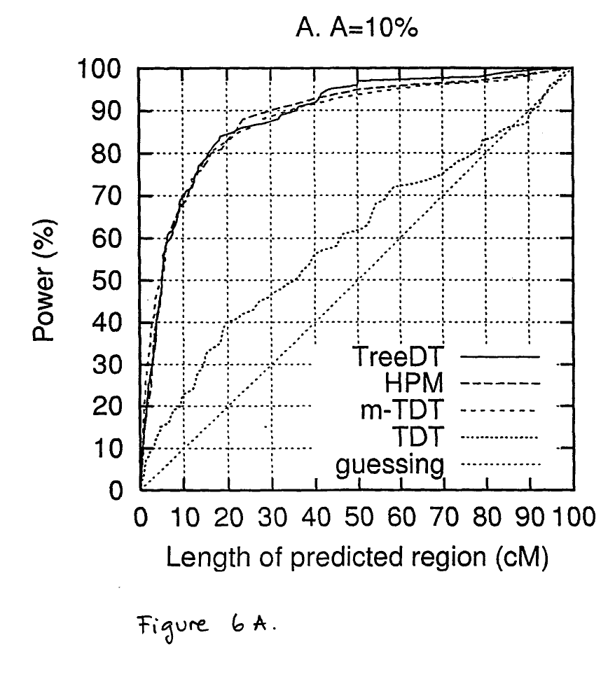

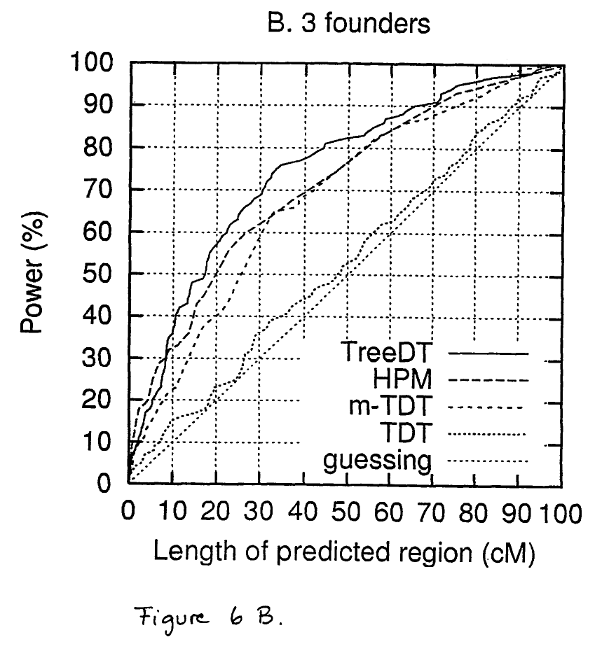

First we assess the prediction accuracy of TreeDT with different values of A, the proportion of disease-associated chromosomes that actually carry the mutation (FIG. 5A). The results are reported as curves that show the percentage of 100 data sets where the gene is within the predicted region, as a function of the length of the predicted region. Or, in other words, the x coordinate tells the cost a geneticist is willing to pay, in terms of the length of the region to be further analyzed, and the y coordinate gives the probability that the gene is within the region. For A=20% or 15% the accuracy is very good, and with lower values of A the accuracy decreases until with A=5% only in 20-30% of data sets can the gene be localized within a reasonable accuracy of 10-20 cM. We remind the reader that the test settings have been designed to be challenging, and to test the limits of the approach.

Next we evaluate the effect of the only parameter of TreeDT, the number of deviant subtrees that are searched for in each tree. An upper limit of 6 subtrees, used in the previous test, is evaluated against fixed amounts of 1, 2, or 3 subtrees, with a varying number of founders that introduced the mutation (FIG. 5B). As we increase the number of founders, evidence about the gene location becomes more fragmented, and accordingly the performace degrades. While the differences between different numbers of subtrees are not large, it is interesting to note that for each number of founders, the same number of subtrees gives marginally the best result. The upper limit of 6 subtrees gives consistently competitive results, so we continue using it in the following experiments.

Gene mapping studies like the ones imitated in the above tests assume, based on some other analyses, that a disease susceptibility gene is indeed present in the analyzed area. TreeDT has the important advantage over plain gene localization methods that it can also be used to predict whether the analyzed region contains a disease susceptibility gene at all or not. The overall p value TreeDT produces indicates the corrected significance of the best single finding, and by setting an upper limit for its value TreeDT can be used to classify data sets to ones that do or do not contain a gene. For data sets with no gene, TreeDT correctly produces overall p values that are uniformly distributed in [0,1]. So, smaller thresholds for p result in less false positives, but also in less true positives. FIG. 5C shows the experimental relationships between power (ratio true positivites/all positives) and overall p (ratio false positives/all negatives). For higher values of A the classification accuracy is extremely good. For A=5% it is comparable to random guessing, although TreeDT is still able to locate an existing gene adequately in 20-30% of the cases (FIG. 5A).

### Example 3

### Comparison to Other Methods

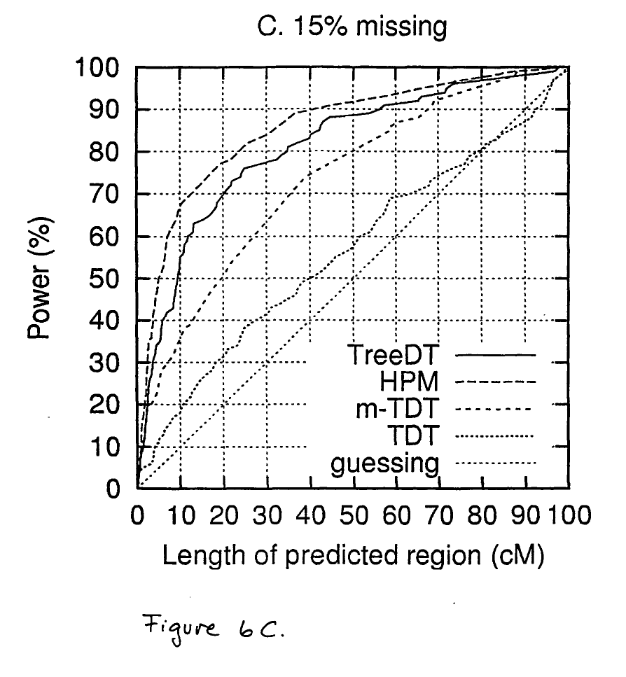

TreeDT, HPM, and m-TDT have practically identical performance in localizing the DS gene in the baseline setting (FIG. 6A). TDT is clearly inferior compared to the other methods. Tests with other values of A give similar results.

In a test setting with three founders who introduced the mutation to the population, differences between the three best methods start to appear (FIG. 6B). TreeDT has an edge over HPM, which in turn has an edge over m-TDT. TDT barely beats random guessing.

Finally, we compare the methods with a large amount of missing data (FIG. 6C). Expectedly, HPM is most robust with respect to missing data since it allows gaps in its haplotype patterns. Surprisingly, TreeDT is not much weaker than HPM, although no actions have been taken in it to account for missing or erroneus data. Performance of m-TDT degrades much more clearly.

Method to method comparisons (not shown) indicate that the prediction errors are mostly caused by random effects in population history—since different methods tend to make mistakes in the same data sets—rather than by systematic differences between the methods. However, those cases where one method succeeds and another fails will give useful input for further improvements of the methods.

The execution time of TreeDT for a single dataset is about ten minutes using 1,000 permutations on a 450 MHz Pentium II. The respective time for HPM with permutations is over 20 minutes.

### REFERENCES

[1] R. Agrawal, T. Imielinski, and A. Swami. Mining association rules between sets of items in large databases. In P. Buneman and S. Jajodia, editors, Proceedings of 1993 ACM SIGMOD Conference on Management of Data, pp 207-216. ACM, Washington, D.C., May 1993.
[2] R. Agrawal, H. Mannila, R. Srikant, H. Toivonen, and A. Verkamo. Fast Discovery of Association Rules. In U. Fayyad, G. Piatetsky-Shapiro, P. Smyth, and R. Uthurusamy, editors, Advances in Knowledge Discovery and Data Mining, pp 307-328. AAAI Press, Menlo Park, Calif., 1996.
[3] B. Devlin, N. Risch, and K. Roeder. Disequilibrium Mapping: Composite Likelihood for Pairwise Disequilibrium. Genomics, 36:1-16, 1996.
[4] L. Kruglyak, M. Daly, M. Reeve-Daly, E. Lander. Parametric and Nonparametric Linkage Analysis: a Unified Multipoint Approach. Am J Hum Genet, 58:1347-1363, 1996.
[5] L. Lazzeroni. Linkage Disequilibrium and Gene Mapping: an Empirical Least-Squares Approach. Am J Hum Genet, 62:159-170, 1998.
[6] M. McPeek and A. Strahs. Assessment of Linkage Disequilibrium by the Decay of Haplotype Sharing, with Application to Fine-scale Genetic Mapping. Am J Hum Genet, 65:858-875, 1999.
[7] A. Nakaya, H. Hishigaki, and S. Morishita. Mining the Quantitative Trait Loci Associated with Oral Glucose Tolerance in the Oletf Rat. Proc. of Pacific Symposium on Biocomputing, pp 367-379, Jan. 4-9, 2000.
[8] S. Service, D. Temple Lang, N. Freimer, and L. Sandkuijl. Linkage-Disequilibrium Mapping of Disease Genes by Reconstruction of Ancestral Haplotypes in Founder Populations. Am J Hum Genet, 64:1728-1738, 1999.
[9] P. Sevon, V. Ollikainen, P. Onkamo, H. Toivonen, H. Mannila, and J. Kere. Mining Associations Between Genetic Markers, Phenotypes and Covariates. Genetic Analysis Workshop 12, Genetic Epidemiology, 21 (Suppl. 1), 2001. In press.
[10] P. Sevon, H. Toivonen, V. Ollikainen. TreeDT: gene mapping by tree disequilibrium test (extended version). Report C-2001-32, Department of Computer Science, University of Helsinki, Finland, 2001.
[11] R. Spielman, R. McGinnis, W. Ewens. Transmission Test for Linkage Disequilibrium: The Insulin Gene Region and Insulin-Dependent Diabetes Mellitus (IDDM). Am J Hum Genet, 52:506-516, 1993.
[12] J. Terwilliger, M. Speer, J. Ott. Chromosome-Based Method for Rapid Computer Simulation in Human Genetic Linkage Analysis. Genetic Epidemiology, 10:217-224, 1993.
[13]J. Terwilliger. A Powerful Likelihood Method for the Analysis of Linkage Disequilibrium Between Trait Loci and One ore More Polymorphic Marker Loci. Am J Hum Genet, 56:777-787, 1995.
[14] H. Toivonen, P. Onkamo, K. Vasko, V. Ollikainen, P. Sevon, H. Mannila, M. Herr, and J. Kere. Data Mining Applied to Linkage Disequilibrium Mapping. Am J Hum Genet, 67:133-145, 2000.
[15] H. Toivonen, P. Onkamo, K. Vasko, V. Ollikainen, P. Sevon, H. Mannila, and J. Kere. Gene Mapping by Haplotype Pattern Mining. Proc. Bio-Informatics and Biomedical Engineering, pp 99-108, Arlington, Va., Nov. 8-10, 2000.

1. A method for gene mapping to discover a gene or DNA region affecting a certain trait using chromosome and phenotype data, which method utilizes linkage disequilibrium between genetic markers mᵢ, which are polymorphic nucleic acid or protein sequences or strings of single-nucleotide polymorphisms deriving from a chromosomal region, which method comprises following steps:
i) identifying a prefix tree T based on the observed haplotypes at a number of locations of a chromosome,
ii) evaluating each prefix tree T by its genetic and statistical feasibility, assuming that the gene was close to the root of the tree, and thus determining a score for each prefix tree T,
iii) predicting the area for the location of the gene as a function of the score determined in the step (ii).

2. The method according to claim 1, wherein in the step (i) the prefix tree T is build between each pair of consecutive markers.

3. The method according to claim 1 or 2, wherein the prefix tree T is build using a string-sorting algorithm.

4. A method according to claim 1, wherein the prefix tree T is evaluated by tree disequilibrium test testing the alternative hypothesis The distribution of the disease-association statuses deviates in some subtrees of T from the overall distribution of statuses against the null hypothesis The disease-association statuses are randomly distributed in the leaves of T.

5. A method according to claim 4, wherein for measuring the disequilibrium of a tree a test statistic Zₖ for a tree with k deviant subtrees T₁, . . . , Tₖ is calculated by the following formula:
$${Z}_{k} = {\sum }_{i = 1}^{k} \frac{{a}_{i} - {n}_{i} p}{\sqrt{{n}_{i} p ( 1 - p )}} ,$$
where aᵢ is the number of disease-associated haplotypes and nᵢ the total number of haplotypes in subtree TᵢεS, S being the given subtree set, and p is the proportion of disease-associated haplotypes in the sample.

6. A method according to claim 4 or 5, wherein the following algorithm is used:
Input: A haplotype prefix tree T
Output: Maximum values of Zₖ in the tree T for each k
Call Maximize(T)
Maximize(T):
If T is not a leaf:
1. For each immediate subtree Tᵢ of T: Recursively call Maximize(Tᵢ).
2. For each k: calculate the maximum value Z_{MAX, k}(T) for Zₖ(S,T) over all S that can be obtained by combining subtree sets from each subtree Tᵢ of T.
3. Calculate Z₁ for T. If Z₁>Z_{MAX, 1}(T) then set Z_{MAX, 1}(T): =Z₁.
If T is a leaf, then set Z_{MAX, 1}(T): =0.

7. A method according to claim 6, wherein step 2 is further refined as follows:
2.1 Set Yₖ: =0 and Z_{MAX, k}(T): =0 for all k, 1≦k≦n, where n is the number of leaves in T.
2.2 For each subtree T′ of T:
2.2.1 For each pair (i,j), 1≦i≦p and 1≦j≦q, where p is the number of leaves in T′ and q is the total number of leaves in all the subtrees processed prior to T′:
If Z_{MAX, i}(T)+Yⱼ>Z_{MAX, i+j}(T), then set Z_{MAX, i+j}(T): =Z_{MAX, i}(T′)+Yⱼ.
2.2.2 For each k, 1≦k≦p:
If Z_{MAX, k}(T)>Z_{MAX, k}(T), then set Z_{MAX, k}(T): =Z_{MAX, k}(T′).
2.2.3 For each k, 1≦k≦p+q:
If Z_{MAX, k}(T)>Yₖ(T), then set Yₖ(T): =Z_{MAX, k}(T)

8. A method according to any of claims 4 to 7 wherein the significance of the disequilibrium at a given location is tested by multiple nested permutation test.

9. A method according to claim 8, wherein the permutation test comprises following steps:
finding for each k the set S of subtrees that maximizes Zₖ and estimating the p value for each maximized Zₖ
estimating a new p value for a combination of the information from the prefix tree T to the left and to the right of the location, combined measure being the product of the lowest p value over all k, and ranking locations by the new p values,
obtaining the point prediction for the gene location by taking the best location from the p value ranked list of locations and obtaining a single corrected p value for the best finding with a test using the lowest local p value as the test statistic.

10. A method according to claim 9, wherein the following algorithm is used:
1. Compute Z_{MAX, k}(T)=max Zₖ(T,S) for each subtree count k and each coalescence tree T over all SεSubtreeSets(T).
2. Randomly generate n+1 permutations of disease-association statuses for the haplotypes and for each permutation i and (T,k): compute Z_{MAX, k}(i,T)=max Zₖ(i,T,S) over all SεSubtreeSets(T).
//Level 1
3. For each (T,k):
3.1 Calculate a p value p(T,k) by comparing Z_{MAX, k}(T) to Z_{MAX, k}(i,T), 1≦i≦n.
3.2 For each permutation i: calculate a p value p(i,T,k) by comparing Z_{MAX, k}(i,T) to all Z_{MAX, k}(j,T), j≠i.
//Level 2
4. For each pair of opposed trees rooted at the same location t=(T₁,T₂):
4.1 Choose p_{MIN}(t)=min p(T₁,k₁)p(T₂,k₂) over all k₁, k₂
4.2 For each permutation i: choose p_{MIN}(i,t)=min p(i,T₁,k₁)p(i,T₂,k₂) over all k₁, k₂.
4.3 Calculate a p value p(t) by comparing p_{MIN}(t) to p_{MIN}(i,t), 1≦i≦n.
4.4 For each permutation i: calculate a p value p(i,t) by comparing p_{MIN}(i,t) to all p_{MIN}(j,t), j≠i.
//Level 3
5. Choose p_{MIN}=min p(i,t) over all t.
6. For each permutation i: choose p_{MIN}(i)=min p(i,t) over all t.
7. Calculate the overall corrected p value by comparing p_{MIN} to p_{MIN}(i), 1≦i≦n.

11. A computer-readable data storage medium having computer-executable program code stored thereon operative to perform a method of any of preceding claims when executed on a computer.

12. A computer system programmed to perform the method of any of claims 1-10.
